(19) United States
(12) Patent Application Publication
(10) Pub. No.: US 2012/0203113 A1
(43) Pub. Date: Aug. 9, 2012

(54) BLOOD FLOW SENSOR

(76) Inventors: Olaf Skerl, Bad Doberan (DE); Michael Lippert, Ansbach (DE)

(21) Appl. No.: 13/350,855

(22) Filed: Jan. 16, 2012

Related U.S. Application Data
(60) Provisional application No. 61/438,985, filed on Feb. 3, 2011.

(51) Int. Cl. A61B 6/00; A61N 1/365

(52) U.S. Cl. 600/473; 600/479; 607/18

(57) ABSTRACT

A blood flow rate sensor has at least one transmitter for emitting waves into a blood vessel, the propagation of which is deflected by cellular blood components, and at least two receiver units for receiving waves emitted by the transmitter. The receiver units are spaced from each other in the direction of blood flow, and are situated such that each receives waves from a different path through the blood. The output signal of each receiver unit is filtered or otherwise processed to obtain a noise component, and the noise components from the receiver units are cross-correlated to determine a time offset between the output signals. The time offset is inversely proportional to the blood flow rate.

### CROSS-REFERENCE TO RELATED APPLICATIONS

This patent application claims the benefit of U.S. Provisional Patent Application No. 61/438,985, filed on Feb. 3, 2011, which is hereby incorporated by reference in its entirety.

### FIELD OF THE INVENTION

The invention relates to an implantable blood flow sensor for measuring a blood flow rate of a living body, i.e., the flow velocity of the blood in a blood vessel.

### BACKGROUND OF THE INVENTION

The blood flow rate (also simply referred to as “blood flow” or “flow rate”) in selected blood vessels is an important diagnostic parameter. As an example, the effective stroke volume may be determined from the blood flow in the aorta, and based on the variation of the aortal flow over time, conclusions may be drawn concerning the rate of cardiac insufficiency, the hemodynamic effects of arrhythmia, possible treatment parameters, and/or the vascular characteristics of the aorta. As another example, the blood flow in the renal arteries is an important parameter for the diagnosis and treatment of renal insufficiency.

Numerous methods are known, and in some cases used, for measuring the blood flow in blood vessels. Acoustic methods, primarily based on the Doppler effect (ultrasound Doppler sonography), are generally known and widely used. A disadvantage of using acoustic Doppler measurement in implantable systems is the requirement for precise and consistently stable alignment of the acoustic beam on the blood vessel to be investigated. Slight deviations or fluctuations may result in measuring errors which cannot be compensated for. In addition, the Doppler method preferably directs the acoustic beam at the flattest angle possible with respect to the vessel, which makes positioning of the beam emitter more difficult. Approaches are also known wherein the acoustic beam extends perpendicular to the vessel. For example, in U.S. Pat. No. 5,785,657, the autocorrelation function (ACF) of the received signal is determined for this purpose. The minima of the ACF are used to determine the time required for the scattered particles to cross the measured volume. If the dimensions of the measured volume are precisely known, the velocity and thus the flow rate of the particles may be determined. Again, a disadvantage of these methods is that the resulting measured volume must be precisely known and consistently stable. Methods based on the ultrasound travel time process are also used, in which two acoustic transducers are externally applied to the blood vessel in an offset manner. Again, a disadvantage is the requirement for precise alignment of the acoustic transducers, and for exact knowledge (and long-term stability) of the distance between the transducers. In addition, this method requires high-precision measurement of the travel time of the acoustic pulses with accuracies of less than 1 nanosecond (ns), which is difficult to achieve with long-term implants.

Also generally known are optical methods based on the principle of the laser Doppler anemometer, which are disadvantageous for use with long-term implants owing to their high energy requirements and relatively high level of technical complexity. Alternative optical methods, for example according to U.S. Pat. No. 5,601,611, use complex computing methods to directly evaluate signals back-scattered by the blood components in order to obtain information concerning the blood flow rate. Here as well, the high level of technical complexity is disadvantageous for use with long-term implants. Methods are also known which use the photoplethysmogram (PPG) for determining surrogate parameters for blood pressure and blood flow. For example, the PPG sensor described in U.S. 2010/049060 is located on the housing of an implantable medical device (IMD), preferably in the header thereof. The document describes methods wherein surrogate parameters for the blood pressure and the blood flow may be determined based on the PPG. A disadvantage of these methods is that they are based on assumed blood flow models and parameters which are not precisely known, and which are subject to time-related and individual fluctuations. In addition, secondary effects such as changes in the vascular tone, for example, have a great influence on the determination of these surrogate parameters.

Thermal methods represent another major class of methods for blood flow measurement. Thermodilution processes, for example, are generally used, although the classical methods of so-called hot-wire anemometry are also prevalent. Also known are correlation methods in which a thermal pattern is applied to the blood flow, and the travel time thereof over a defined distance is measured. However, for all thermal methods, high energy requirements and the need for distinct, localized introduction of heat into the blood are disadvantageous.

The use of so-called micro-hair sensors for blood flow measurement is also known. Micro-hairs or banners manufactured using MEMS technology project into the blood flow, thereby being bent or deflected; see U.S. 2008/0154141, for example. A disadvantage of these arrangements is that clots may form on the foreign bodies protruding into the blood flow. In addition, the micro-hairs or banners may become agglutinated or encapsulated, thus losing their function.

### SUMMARY OF THE INVENTION

The invention involves blood flow sensors which offer alternatives to the foregoing flow sensors. An exemplary version of a blood flow sensor for measuring a blood flow rate in a blood vessel of a living body has the following components:
a support bearing at least one transmitter for emitting waves, wherein the propagation of the waves is deflected by cellular constituents of the blood with respect to the propagation in the blood plasma;
two receiver units attached to the support at a distance from one another for receiving waves emitted by the transmitter, wherein the receiver units are situated in such a way that during use, the received waves for the different units have followed a different path through the blood, with the receiver units preferably being offset with respect to one another at least in the direction of flow of the blood;
a filter unit connected to the receiver units for filtering an output signal of a respective receiver unit in such a way that a particular filtered output signal of the filter unit represents a noise component of a particular output signal of a particular receiver;
a cross-correlation unit connected to the filter unit for determining a time offset between the filtered output signal of one receiver unit and the filtered output signal of the other receiver unit by correlation of the filtered output signal of one receiver unit with the filtered output signal of the other receiver unit, the time offset being inversely proportional to the particular blood flow rate.

The support and the transmitter and receiver units attached thereto together preferably form an implantable sensor system.

A measurement principle basically known from U.S. Pat. No. 3,762,221 may also be used for determining the blood flow rate, based on the properties and the composition of the blood.

It is known that the damping of light in the blood, corresponding to the damping characteristic of the blood, differs as a function of the wavelength. The damping characteristic of the blood is also a function of its oxygen loading. This property is used in photoplethysmography (PPG) in order to determine the partial oxygen saturation of the blood, by measuring the ratio of the light dampings at two given wavelengths. It is known that a photoplethysmogram may also be used to derive the blood pressure curve, for example due to the influence of the blood pressure wave on the expansion of the blood vessels.

In addition, blood may be considered in simplified terms as an emulsion in which the blood components (erythrocytes, leukocytes, for example) are statistically distributed. This results in noise which is superimposed on the photoplethysmogram signal. For conventional applications, this noise is suppressed using signal processing measures, for example filtering or averaging of multiple curves. The particular noise pattern results from the spatial distribution of the blood components over the vessel cross section. The inventors have found that the spatial distribution of the blood components in the flow, and therefore also the noise pattern caused thereby, is primarily maintained over a small distance. The inventors have also found that the shift of the noise pattern over time may be determined by recording a photoplethysmogram at two closely spaced locations, and removing the noise component by cross-correlation of the two noise components. If the distance between the measuring sites is known, the flow rate may be calculated using the time offset of the noise pattern.

The inventors have also found that a blood flow rate measurement of this nature may be carried out independently of photoplethysmography, as by using the aforementioned exemplary blood flow sensor according to the invention.

The blood flow sensor according to the invention preferably involves an implantable sensor system for determining the blood flow in a blood vessel. The sensor system is preferably attached to an elastic cuff bearing the sensors, which is to be placed around a blood vessel. Such a cuff is also referred to below as a sensor cuff. An implantable medical device (IMD) having an electronics system for controlling the sensors and evaluating the sensor signals, a power supply, and a telemetry unit may likewise be situated on the cuff or connected thereto via a cable, and may have an offset housing. At least two preferably identical or at least similar sensor systems are mounted on the elastic cuff at a suitable defined distance (1 cm, for example). The cuff itself may also be used as a pressure sensor by designing it to determine the expansion of the vascular wall according to known methods. As a result of fixedly mounting the sensor systems on the cuff, the geometric distance between the sensor systems is sufficiently stable, even over a long period of time.

The sensor system of the invention preferably has the following components:
Transmitters having at least two light sources of suitable wavelength (for example, 660 nm for red and 910 nm for infrared) and an oppositely situated optical receiver unit for measuring the irradiation of the blood flow; or
Transmitters having at least two light sources of suitable wavelength (for example, 660 nm for red and 910 nm for infrared) and an angularly offset optical receiver unit, which is not oppositely situated, for measuring the reflection/scattering by blood components in the bloodstream; or
Transmitters having a light source of suitable wavelength (for example, 660 nm for red) and an oppositely situated optical receiver unit for measuring the irradiation; or
Transmitters having a light source of suitable wavelength (for example, 660 nm for red) and an angularly offset optical receiver unit, which is not oppositely situated, for measuring the reflection/scattering.

As light sources, suitable light-emitting diodes (LEDs) may be directly mounted on the cuff, or light from a light source can be supplied via a light pipe such as an optical fiber (illuminating fiber) or other light-carrying element. When an optical fiber is used, it is possible to link the illuminating fibers of the individual sensor systems to a common LED, and/or to inject multiple wavelengths (multiple LEDs) into the illuminating fiber at time intervals. The linking of the sensor cuff to the implantable medical device (IMD) via optical fibers or other light pipes has the advantage that there is no electrically conductive connection between the IMD and the sensor cuff. However, the use of light pipes can result in greater light loss, which must be compensated for by higher energy expenditure.

An optical receiver unit may contain a suitable converter element, for example a photodiode, which converts optical signals to electrical signals. These output signals of the receiver units, or of the converter elements of the receiver units, are also referred to below as reception signals. The optical receiver unit may also be connected to the sensor system via an optical fiber (receiving fiber) or other light pipe, with a separate receiving fiber preferably being used for each reception channel.

It is also possible to use a single fiber or other light pipe for the illumination and the reception, with the injected beams and extracted beams being separated using suitable means, for example semitransparent mirrors. However, any losses introduced by such separation means must be compensated for by a higher expenditure of energy.

In addition, the beam geometry may be favorably influenced using suitable optical means, for example slit diaphragms, in order to eliminate interferences from the adjacent sensor system, to design the measurement path in a locally delimited manner, and/or to attain other benefits.

The reception signals are used to determine the ratio of the dampings (i.e., reflected portions) at the various wavelengths or the damping (reflected portion) at one wavelength, or only the reception signal itself is used as a raw signal for the further signal processing. Based on this raw signal, the noise component produced by the blood components is then removed, using known signal processing means, for example by filtering using a band pass filter, thus obtaining a corresponding noise signal for each reception channel. The PPG signal which likewise results when multiple wavelengths are used is not needed for the approach according to the invention, but it may be provided if needed for other diagnostic uses. Based on the two noise signals, the time offset between the two noise signals is determined by cross-correlation using known methods. Since the geometric distance between the sensor systems is known, it is used to determine the magnitude as well as the direction of the blood flow rate.

The invention is not limited to the use of light waves, and may be implemented using types of waves whose propagation is damped by the blood components, or which are reflected or scattered by the blood components, for example ultrasound waves or electromagnetic waves.

The values of the flow rate may be averaged synchronously with the cardiac cycle over a given number of cardiac cycles in order to suppress interferences. Suitable parameters may be derived from the flow rate (for example, average values, diastolic value, systolic value, difference between the diastolic and the systolic value, increases in the blood flow curve at specific times in the cardiac cycle, and so forth).

The flow rate or the derived parameters may be used to determine trends or trend parameters, which may be compared to threshold values in order to generate alarms, for example.

Relationships between the heart rate and the blood flow rate may also be determined. In addition to the possibility for providing the two sensors in a cuff around a blood vessel, two sensors may also be placed on a catheter or an implantable electrode in the bloodstream (for measuring reflection/scattering). This allows blood flow measurement, for example, in the vena cava, in the coronary sinus, or between the right atrium and the right ventricle through the plane of the cardiac valve (mitral flow).

The blood flow sensor is preferably connected to a control and evaluation unit which is designed to evaluate values of the blood flow rate in combination with further parameters. The following combinations, for example, are particularly preferred:

In combination with an electrocardiogram (ECG):
Time intervals between electrical excitation of the heart and the resulting blood flow;
Triggering of the blood flow measurement by specific conditions, for example tachycardia.

In combination with an acceleration sensor:
Measurement of the blood flow during activity or rest, differences between the corresponding blood flow rates;
Measurement while the patient is standing or lying, differences between standing and lying;
Recording the heart sounds and relationships between heart sounds and blood flow.

In combination with a pressure sensor: relationships between blood pressure and blood flow to obtain information concerning, for example, the condition of the blood vessels.

Implantable medical devices (IMD) which are designed to record an electrocardiogram and/or which have an acceleration sensor are well known.

Using a telemetry unit which is integrated into the blood flow sensor (or into an implantable or other medical device connected to the blood flow sensor), data transmission to an external device, for example a patient device, is possible. This in turn allows telemonitoring and/or the display and evaluation of detected data and data combinations in a central service center, and thus also allows incorporation of the data into a predictive model. A large amount of data, including data originating from blood flow sensors or implantable medical devices of various patients, may be evaluated in combination in a central service center, thus allowing better assessment of the data and more accurate diagnoses and/or predictions.

Possible applications or refinements of the blood flow sensor include the following:
Monitoring of the perfusion of organs, for example the kidneys;
Monitoring of the effects/side effects of medications and treatment optimization;
As a hemodynamic sensor, for example for CRT treatment optimization, or as an additional parameter for a heart defect predictive model;
In combination with the electrocardiogram (ECG): determination of the electromechanical coupling in the heart, and based on such determinations, optimization of treatment parameters (AV, VV delay, for example);
Determination of the hemodynamic effects of tachycardia or detection of tachycardia, for example as an additional shock criterion, or for minimizing delivered shocks (for example, when sufficient blood flow is detected despite tachycardia);
Based on the mitral flow, parameters which are equivalent to the E- and A-waves from echocardiography (which may be used, for example, to optimize CRT treatment);
Evaluation of the signal parameters of the noise itself, for example estimation of the hematocrit value or parameters for the size/size distribution of the blood components.

### BRIEF DESCRIPTION OF THE DRAWINGS

Exemplary versions of the invention are explained below in greater detail with reference to the accompanying figures, which show the following:

FIG. 1: shows a schematic diagram of an exemplary blood flow sensor according to the invention;

FIGS. 2A and 2B: show two schematic diagrams (side view and cross section) of two sensor systems on an elastic cuff for a blood flow measurement by irradiation;

FIGS. 3A and 3B: show two schematic diagrams (side view and cross section) of two sensor systems on an elastic cuff for a blood flow measurement by detection of scattered radiation;

FIG. 4: shows a schematic diagram of a sensor system in the form of a catheter which may be introduced into a blood vessel; and

FIG. 5: shows the different damping of light of various wavelengths at various blood oxygen saturations.

### DETAILED DESCRIPTION OF EXEMPLARY VERSIONS OF THE INVENTION

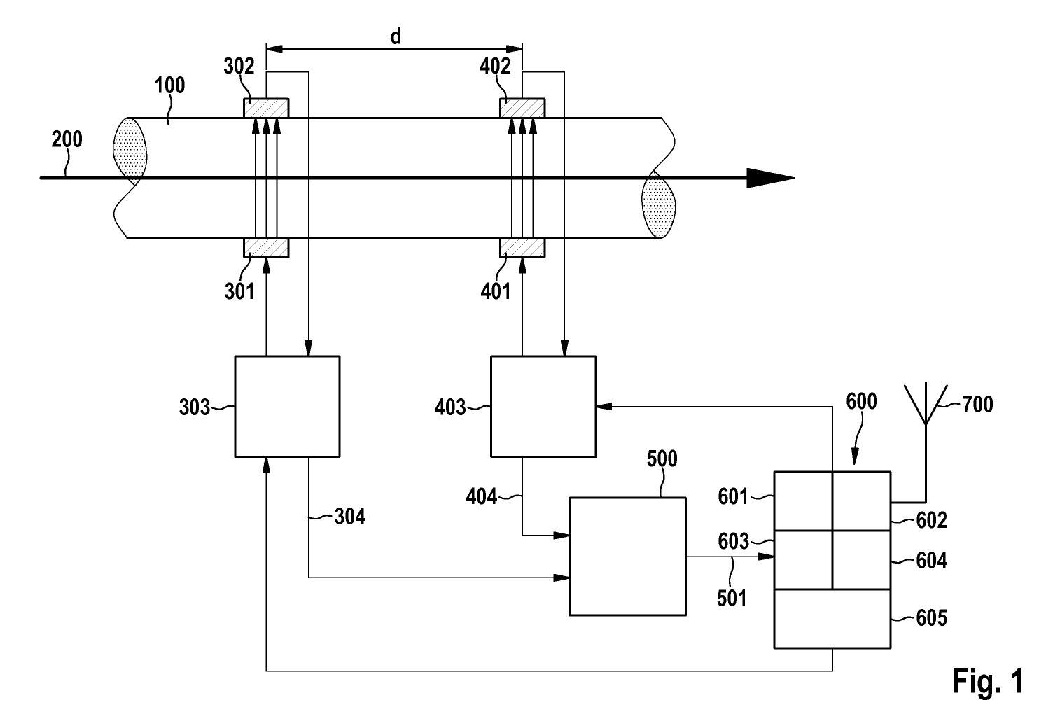

FIG. 1 shows an exemplary version of a blood flow sensor according to the invention. This blood flow sensor is used to determine the velocity of a blood flow 200 in a blood vessel 100. For this purpose, two sensor systems, each having a transmitter 301 or 401 and a receiver unit 302 or 402, are provided. The receiver unit 302 of the first sensor system is situated on a side of the blood vessel 100 opposite from the transmitter 301 of the first sensor system, so that light (e.g., red or infrared light) emitted by the transmitter 301 transversely irradiates the blood flow 200 and then strikes the receiver unit 302. The second sensor system having transmitter 401 and receiver unit 402 has a similar design, and is offset with respect to the first sensor system by a distance d in the longitudinal direction of the blood vessel 100. A distance d in the range of several mm to several cm is suitable.

Each transmitter 301 or 401 contains a light source, for example a light-emitting diode (LED), for emitting light. The light sources do not always have to be situated directly adjacent to the blood vessel 100. The light source may also inject light into the blood vessel 100 via an optical fiber (not illustrated in FIG. 1).

Each receiver unit 302 or 402 has a converter element which is designed for receiving light emitted by a respective transmitter and converting the light to an electrical output signal, also referred to as a reception signal. A photodiode, for example, is a suitable converter element. Such a converter element also does not necessarily have to be situated directly adjacent to the blood vessel 100, but instead may likewise be optically coupled to the blood vessel 100 via an optical fiber in order to extract light from the blood vessel 100.

Each of the sensor systems 301/302 and 401/402 is connected to a sensor control and filter unit 303 or 403, respectively. Each sensor control and filter unit 303/403 is designed to control its respective sensor 301 or 401, and to receive and evaluate the respective reception signal of the receiver unit 302 or 402. The conditioning and filtering of each reception signal by its sensor control and filter unit 303/403 includes filtering, preferably filtering by a band pass filter, of the reception signal in order to generate a corresponding sensor output signal 304 or 404 whose signal shape is essentially determined by the noise component of the reception signal.

The sensor output signals 304 and 404 are then supplied to a cross-correlation unit 500, which subjects the sensor output signals 304 and 404 from the two different sensor systems to cross-correlation, the result of which is a time offset between the two sensor output signals, so that the signal features of each sensor output signal appear in a time-shifted form in the other sensor output signal. This time offset corresponds to the period of time needed by the blood flow 200 to travel the distance d by which the two sensor systems 301/302 and 401/402 are offset with respect to one another in the direction of the blood flow 200. The cross-correlation unit 500 thus generates a time shift signal 501 which corresponds to a signal travel time over the distance d through the bloodstream. The time shift signal 501 is supplied to a control and evaluation unit 600 which is designed to calculate for the known distance d the blood flow, i.e., the flow rate of the blood, over the distance d based on the signal travel time. The control and evaluation unit 600 also controls the two sensor control and filter units 303 and 403. For this purpose the control and evaluation unit 600 contains a microcontroller 601 and a telemetry unit 602, by means of which detected values may be transmitted via a telemetry antenna 700 to an external device, or by means of which the control commands may be received from an external device. For this purpose, detected values or control commands may be temporarily stored in a memory 603 of the control and evaluation unit 600. Lastly, a sequence control system 604 contains the control commands which control the operation of the control and evaluation unit 600. Since the control and evaluation unit 600 is designed as an electronic control system, it also has a power supply 605, for example in the form of a battery.

As stated above, the control and evaluation unit 600 may also be connected to further sensors or other control units, so that the signals of the blood flow sensor characterizing the blood flow may be combined and evaluated with other signals, in particular signals of an implantable cardiac stimulator (a cardiac pacemaker or a cardioverter/defibrillator, for example) and/or signals of a pressure sensor.

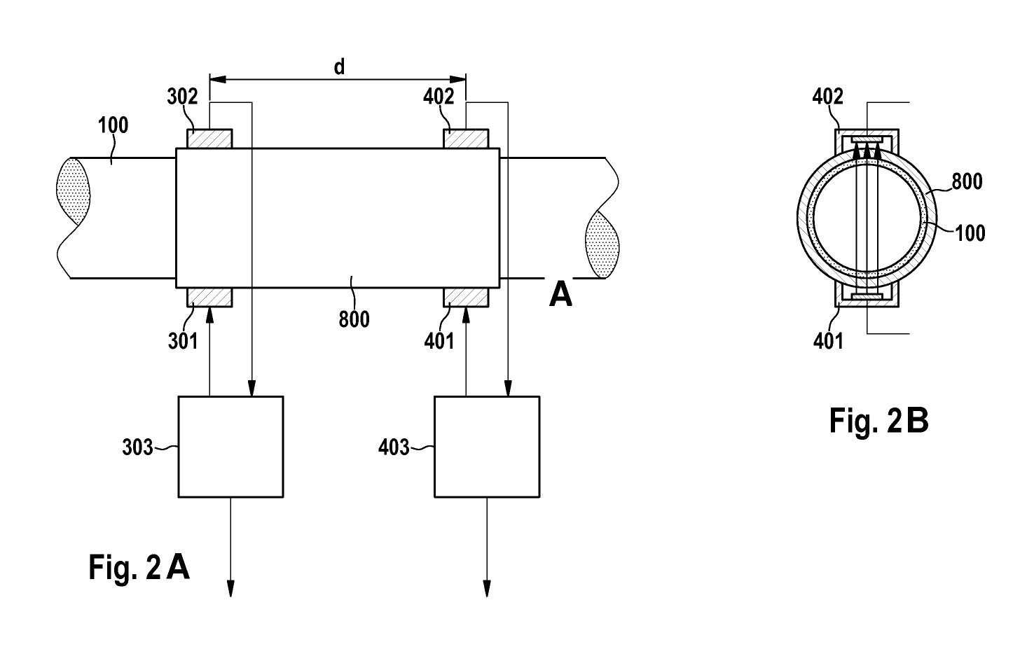

FIG. 2A schematically shows the manner in which the two sensor systems may be fastened at their transmitters 301 or 401 and their receiver units 302 or 402 to an elastic cuff 800, which may be placed around a blood vessel 100. The two sensors are situated on the elastic cuff 800 at a distance d from one another, in particular in such a way that each of the two sensor systems transversely illuminates the blood vessel 100 (see the cross section of FIG. 2B). While not shown, the elastic cuff 800 may be designed as a pressure sensor in which means are provided for generating a pressure signal which represents the pulsing blood flow, based on the elastic deformation of the cuff 800. Such a pressure signal of a pressure sensor may be evaluated together with the blood flow signal.

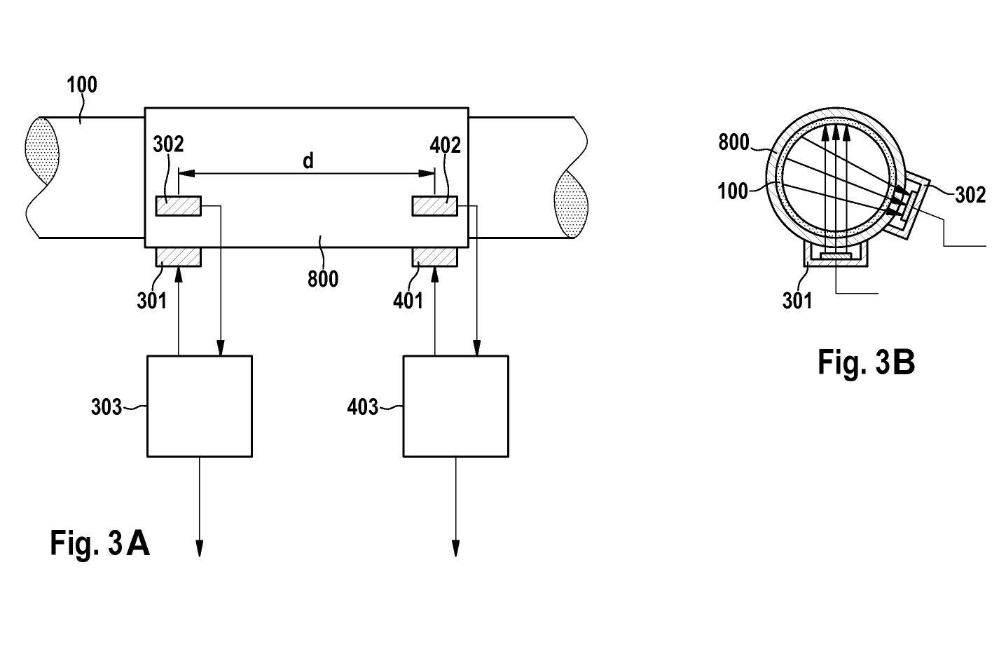

FIG. 3A shows an alternative configuration of the transmitter 301 or 401 and receiver unit 302 or 402 of the sensor system on an elastic cuff 800. In the configuration shown in FIG. 3, each receiver unit 302 or 402 is situated not directly opposite its corresponding transmitter 301 or 401, but rather in such a way that each receiver unit 302 or 402 detects incident light, for example as the result of scattering on blood components, transverse to a direction of irradiation. This is seen particularly clearly in the cross section of a blood vessel 100 in FIG. 3B.

Thus, the sensor system shown in FIGS. 2A-2B detects damping of the light emitted by a respective transmitter by the blood components, whereas the sensor systems shown in FIGS. 3A-3B detect light which is scattered or reflected by the blood components.

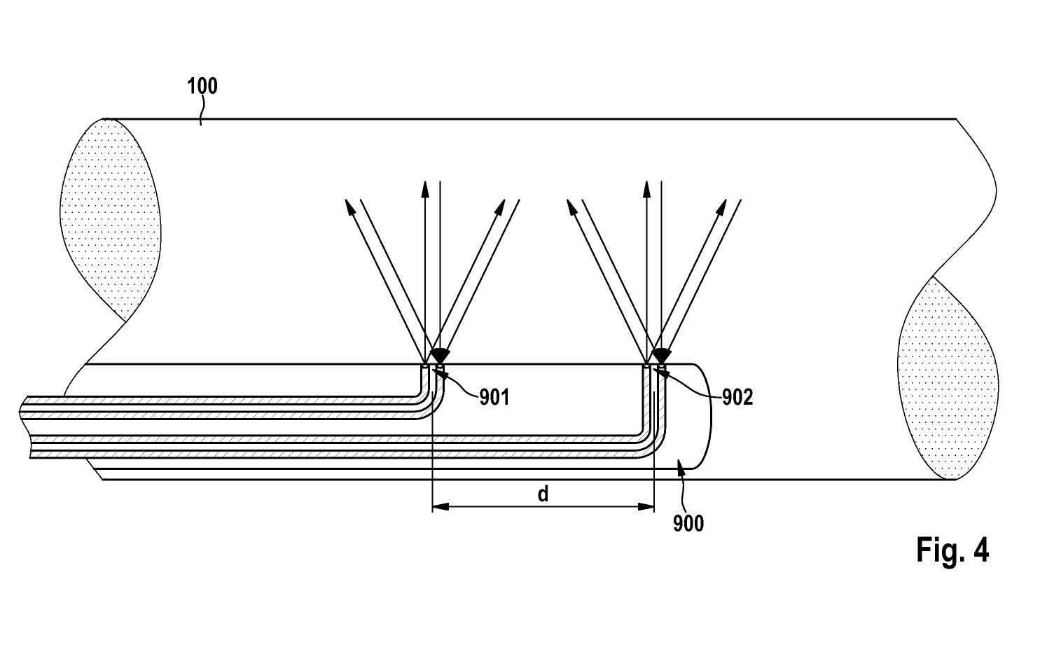

FIG. 4 shows another configuration of the support and the sensor systems in the form of a catheter 900 which is designed for insertion into a blood vessel 100. Light is injected into and extracted from the blood with the aid of optical fibers 901 and 902, which are optically connected to a transmitter or receiver unit of a corresponding blood flow sensor.

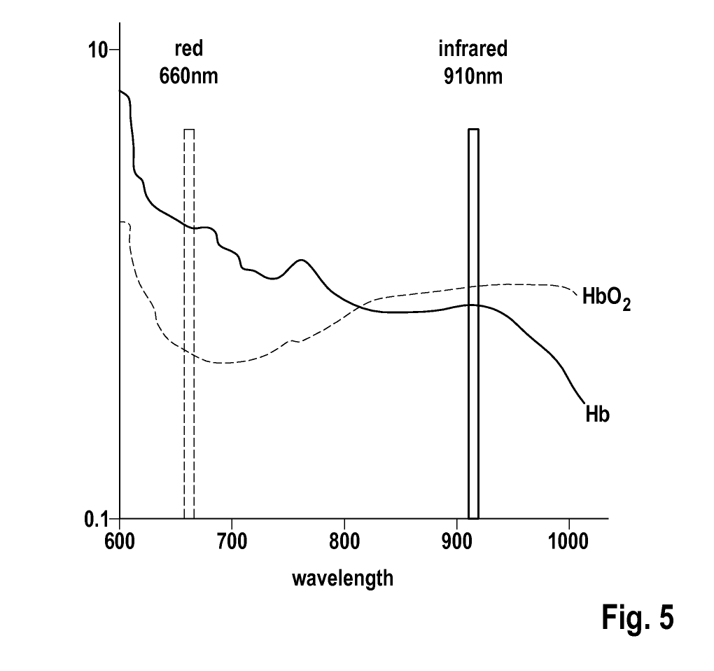

It is particularly advantageous when the blood flow sensor is at the same time designed as a photoplethysmography sensor as described above. In this case, at least one of the sensor systems has at least two transmitters, for example, one emitting light in the red wavelength range (around 660 nm), and the other emitting light in the infrared wavelength range (around 910 nm). FIG. 5 shows the different degrees of damping of light at various wavelengths as a function of the blood oxygen saturation.

The invention's measurement and evaluation of the blood flow rate, alone or preferably in combination with other values, such as blood pressure, blood oxygen saturation, stimulation times, the posture of a patient (standing, sitting, or lying), an activity signal, or the like similarly allows differentiated patient monitoring to be conducted, or improved cardiac pacemaker treatment to be provided. With regard to the latter, it is particularly preferred when the blood flow sensor is a component of a cardiac stimulator, or is connected to a cardiac stimulator.

As an example, the system of FIG. 1 (as described above) can be provided as an elastic vascular cuff connected by cables to an implantable medical device (IMD), for example a pacemaker, an implantable cardioverter/defibrillator (ICD), or a monitoring implant. The IMD contains a power source (a battery, for example), devices for supplying the light sources (for example, light-emitting diodes, also referred to below as LEDs), devices for receiving and filtering the reception signals of the optical receiver unit (photodiodes, for example), devices for controlling the measuring process and for processing the signals, storing the data, and for telemetry with an external patient device. The IMD also contains a device for determining the cross-correlation function based on two reception signals, using generally known methods.

The elastic vascular cuff is equipped with two sensor systems which are mounted at a defined distance from one another (1 cm, for example). Each of these sensor systems contains a light source having two light-emitting diodes (LEDs, for example, for red and infrared light) and an optical receiver unit having a photodiode, for example, which is mounted opposite from the light source. In the first sensor system, the two LEDs in the light source are switched on in alternation at short intervals for a brief period of time (10 ms, for example), and the incident light is measured by the photodiode. Based on this information, the light damping upon passage through the blood flow and then the ratio of the respective light damping at both wavelengths are determined. The signal thus obtained is filtered using a band pass filter (from 200 Hz to 5 kHz, for example), thus removing the noise component of the signal. The second sensor system carries out identical measurement and signal processing at the same time.

The two noise signals of the two sensor systems are supplied to the cross-correlation unit 500 for determining the cross-correlation function. Based on the maxima of the cross-correlation function, the signal processing unit (a microcontroller, for example) determines the time offset of the noise signals, and uses the time offset to determine the blood flow rate based on the known distance between the two sensor systems. The blood flow rates are stored as the blood flow curve over a given period of time, for example a cardiac cycle, in the memory 603 of the signal processing unit (control and evaluation unit 600). Further diagnostic parameters, such as the maximum systolic blood flow or the increase in the blood flow rate in the systole, for example, may be determined from this blood flow curve. These values may also be averaged over multiple cardiac cycles in order to suppress interferences with the blood flow curve. The blood flow curve and/or the parameters determined therefrom may be transmitted at certain intervals via wireless telemetry from the IMD to a patient device, and from there, further transmitted to a telemonitoring service center.

It will be apparent to those skilled in the art that numerous modifications and variations of the foregoing versions of the invention are possible in light of the foregoing discussion. The versions of the invention described above are merely exemplary, and the invention is not intended to be limited to these versions. Rather, the scope of rights to the invention is limited only by the claims set out below, and the invention encompasses all different versions that fall literally or equivalently within the scope of these claims.

What is claimed is:

1. A blood flow rate sensor including:
a. a transmitter configured to emit waves into a blood vessel, wherein the propagation of the waves is affected by blood components within the blood vessel,
b. a pair of receivers spaced from each other along the direction of blood flow, wherein each receiver receives waves emitted by the transmitter along a different path through the blood flow than the other receiver,
c. a filter configured to:
(1) receive an output signal from each receiver, and
(2) isolate a noise component therefrom,
d. a processor configured to determine a time offset between the noise components.

2. The blood flow rate sensor of claim 1 wherein the processor is configured to provide a blood flow rate output signal inversely proportional to the time offset.

3. The blood flow rate sensor of claim 2 further including a control and evaluation unit configured to average blood flow rate output signals over multiple cardiac cycles.

4. The blood flow rate sensor of claim 1 wherein:
a. the transmitter is configured to emit light waves, and
b. the receivers are configured to:
(1) receive an optical signal, and
(2) convert the received optical signal to an electrical signal.

5. The blood flow rate sensor of claim 4 wherein the transmitter includes a light-emitting diode.

6. The blood flow rate sensor of claim 1 wherein the transmitter is configured to emit at least one of:
a. red light having one or more wavelengths at or about 660 nm, and
b. infrared light having one or more wavelengths at or about 910 nm.

7. The blood flow rate sensor of claim 1 wherein the transmitter includes:
a. a light source, and
b. a light pipe optically coupled to the light source.

8. The blood flow rate sensor of claim 1 wherein each receiver contains a photodiode.

9. The blood flow rate sensor of claim 1 including two transmitters, wherein the receivers are each situated to at least primarily receive waves emitted by a respective one of the transmitters.

10. The blood flow rate sensor of claim 1 including two transmitters, wherein:
a. the transmitters are configured to emit light of differing wavelengths,
b. each receiver is:
(1) situated to receive waves emitted by at least one of the transmitters, and
(2) connected in communication with an evaluation unit configured to:
(a) receive an output signal from each receiver, and
(b) determine therefrom a photoplethysmography signal dependent on a ratio of an output signal from light at one wavelength to an output signal from light at another wavelength.

11. The blood flow rate sensor of claim 1 wherein the transmitter and receivers are fixed to a common support.

12. The blood flow rate sensor of claim 11 wherein the support is an elastic cuff configured to be placed at least partially around a blood vessel.

13. The blood flow rate sensor of claim 12 wherein the elastic cuff bears an expansion sensor configured to provide an expansion signal which represents an elastic expansion of the cuff.

14. The blood flow rate sensor of claim 11 wherein the support is a catheter configured for introduction into a blood vessel.

15. The blood flow rate sensor of claim 1 wherein the filter is configured to pass a band of frequencies corresponding to a frequency range of the noise components of the receiver output signals.

16. The blood flow rate sensor of claim 1 in combination with an implantable medical device in communication with the blood flow rate sensor, wherein the implantable medical device includes at least one of:
a. a telemetry unit configured to transmit a signal dependent on the time offset,
b. a cardiac monitoring unit configured to capture electrical signals from a heart, and
c. a cardiac stimulation unit configured to deliver electrical stimulation to a heart.

17. A blood flow rate sensor including:
a. a transmitter configured to emit waves into a blood vessel,
b. a pair of receivers aligned to receive waves emitted by the transmitter, wherein each receiver provides a receiver output signal dependent on the waves received by the receiver,
c. a support maintaining the receivers:
(1) in spaced relationship, and
(2) spaced from the transmitter,
d. a processor configured to provide a blood flow rate output signal dependent on differences between the receiver output signals or components thereof.

18. The blood flow rate sensor of claim 17 wherein:
a. the processor is configured to determine a time offset between noise components of the receiver output signals, and
b. the blood flow rate output signal is dependent on the time offset.

19. The blood flow rate sensor of claim 17 wherein the support is a cuff:
a. configured to be placed at least partially around a blood vessel, and
b. bearing an expansion sensor thereon, the expansion sensor being configured to provide an expansion signal dependent on the size of a blood vessel within the cuff.

20. A method of sensing blood flow rate including the steps of:
a. emitting waves into a blood vessel, wherein the propagation of the waves is affected by blood components within the blood vessel,
b. receiving waves emitted by the transmitter at a pair of locations spaced from each other along the direction of blood flow, wherein each location receives the waves from a different path through the blood flow than the other location,
c. isolating a noise component from the waves received at each location,
d. determining a time offset between the noise components of the locations, and
e. providing a blood flow rate output signal dependent on the time offset.
